(19) United States
(12) Patent Application Publication
(10) Pub. No.: US 2018/0273695 A1
(43) Pub. Date: Sep. 27, 2018

(54) PROCESSES FOR PRODUCING LIGNIN-BASED ENZYMATIC HYDROLYSIS ENHANCERS, AND COMPOSITIONS PRODUCED THEREFROM

(71) Applicant: API Intellectual Property Holdings, LLC, Minnetrista, MN (US)

(72) Inventors: Vesa PYLKKANEN, Atlanta, GA (US); Theodora RETSINA, Atlanta, GA (US)

(21) Appl. No.: 15/924,539

(22) Filed: Mar. 19, 2018

Related U.S. Application Data
(63) Continuation of application No. 14/951,033, filed on Nov. 24, 2015, now abandoned.
(60) Provisional application No. 62/085,464, filed on Nov. 28, 2014.

(51) Int. Cl. C08H 8/00; C12P 19/02; C12N 9/42; C12P 19/14; C08H 7/00

(52) U.S. Cl. CPC C08H 8/00; C12P 19/02; C12Y 302/01004; C12P 19/14; C08H 6/00; C12N 9/2437

(57) ABSTRACT

This disclosure provides lignin-based enzymatic hydrolysis enhancer that includes ethanol-soluble, partially sulfonated lignin. Some embodiments provide a lignin-based enzymatic hydrolysis enhancer comprising AVAP® lignin. Certain embodiments provide a lignin-based enzymatic hydrolysis enhancer comprising AVAP® lignin and lignosulfonates. In some variations, a process for producing a lignin-based enzymatic hydrolysis enhancer comprises fractionating biomass with an acid, a solvent for lignin, and water, to generate cellulose-rich solids and a liquid containing hemicellulose and lignin; recovering the lignin; and generating a lignin-based enzymatic hydrolysis enhancer comprising the lignin. Surprisingly, the lignin-based enzymatic hydrolysis enhancer is experimentally able to enhance glucose yields by 10% or more.

### PRIORITY DATA

This patent application is a continuation of U.S. patent application Ser. No. 14/951,033, filed Nov. 24, 2015, which claims priority to U.S. Provisional Patent App. No. 62/085,464, filed Nov. 28, 2014, each of which is hereby incorporated by reference herein.

### FIELD

The present invention generally relates to processes for fractionating lignocellulosic biomass into cellulose, hemicellulose, and lignin.

### BACKGROUND

Biomass refining (or biorefining) has become more prevalent in industry. Cellulose fibers and sugars, hemicellulose sugars, lignin, syngas, and derivatives of these intermediates are being utilized for chemical and fuel production. Indeed, we now are observing the commercialization of integrated biorefineries that are capable of processing incoming biomass much the same as petroleum refineries now process crude oil. Underutilized lignocellulosic biomass feedstocks have the potential to be much cheaper than petroleum, on a carbon basis, as well as much better from an environmental life-cycle standpoint.

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network. Some forms of biomass (e.g., recycled materials) do not contain hemicellulose.

Improved processes, systems, and additives are desired for more efficiently hydrolyzing cellulose-rich solids, obtained from various types of biomass pretreatment or fractionation, into glucose.

### SUMMARY

In some variations, the present invention provides a process for producing a lignin-based enzymatic hydrolysis enhancer, the process comprising:
(a) providing a lignocellulosic biomass feedstock;
(b) fractionating the feedstock in the presence of a sulfur-containing acid, a solvent for lignin, and water, to generate cellulose-rich solids and a liquid containing hemicellulose and lignin;
(c) recovering at least some of the lignin from the solvent; and
(d) generating a lignin-based enzymatic hydrolysis enhancer comprising the lignin recovered in step (c).

In some embodiments, the sulfur-containing acid is selected from the group consisting of sulfur dioxide, sulfur trioxide, sulfurous acid, sulfuric acid, sulfonic acid, lignosulfonic acid, and combinations or derivatives thereof.

In some embodiments, the lignocellulosic biomass feedstock is a hardwood or a mixture containing a hardwood.

The lignin-based enzymatic hydrolysis enhancer may further comprise hydrophilic, sulfur-containing lignin derived from the feedstock and the sulfur-containing acid. Alternatively, or additionally, the lignin-based enzymatic hydrolysis enhancer may further comprise lignosulfonates that are not derived from the process.

The process in some embodiments further comprises (i) applying the lignin-based enzymatic hydrolysis enhancer to a mixture comprising cellulose-rich solids and cellulase enzymes, and (ii) enzymatically hydrolyzing the cellulose-rich solids to generate glucose.

The cellulose-rich solids may be obtained from (for example) a biomass-pretreatment process selected from the group consisting of steam explosion, hot-water extraction, solvent extraction, acidic solvent extraction, organosolv, dilute-acid pretreatment, ammonia pretreatment, Kraft pulping, sulfite pulping, soda pulping, mechanical pulping, and combinations thereof.

The lignin-based enzymatic hydrolysis enhancer may be present in the mixture at a concentration of about 1 g/L to about 15 g/L, such as about 2 g/L to about 10 g/L.

At least 5% or at least 10% higher glucose yield is achieved with the lignin-based enzymatic hydrolysis enhancer present in the mixture, compared to an otherwise-identical mixture without the lignin-based enzymatic hydrolysis enhancer, in some embodiments of the invention.

Optionally, the process further comprises recovering hemicellulosic sugars from the hemicellulose.

Other variations provide a lignin-based enzymatic hydrolysis enhancer product produced by a process comprising the steps of:
(a) providing a lignocellulosic biomass feedstock;
(b) fractionating the feedstock in the presence of a sulfur-containing acid, a solvent for lignin, and water, to generate cellulose-rich solids and a liquid containing hemicellulose and lignin;
(c) recovering at least some of the lignin from the solvent; and
(d) generating a lignin-based enzymatic hydrolysis enhancer comprising the lignin recovered in step (c).

Some variations provide a lignin-based enzymatic hydrolysis enhancer composition comprising ethanol-soluble lignin. In some embodiments, the ethanol-soluble lignin is partially sulfonated. In some embodiments, the composition further comprises hydrophilic, sulfur-containing lignin and/or lignosulfonates.

A mixture may include the lignin-based enzymatic hydrolysis enhancer composition, cellulose-rich solids, and cellulase enzymes. In some embodiments, the lignin-based enzymatic hydrolysis enhancer composition is present in the mixture at a concentration of about 1 g/L to about 15 g/L, such as about 2 g/L to about 10 g/L.

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

In some variations, the present invention provides a process for producing a lignin-based enzymatic hydrolysis enhancer, the process comprising:
(a) providing a lignocellulosic biomass feedstock;
(b) fractionating the feedstock in the presence of an acid, a solvent for lignin, and water, to generate cellulose-rich solids and a liquid containing hemicellulose and lignin;
(c) recovering at least some of the lignin; and
(d) generating a lignin-based enzymatic hydrolysis enhancer comprising the lignin.

In some embodiments, the hydrophilic lignin includes sulfonated lignin. The hydrophilic lignin may include sulfonated lignin when, for example, the acid comprises a sulfur-containing acid or a derivative thereof In some embodiments, a sulfur-containing acid or a derivative thereof is selected from the group consisting of sulfur dioxide, sulfur trioxide, sulfurous acid, sulfuric acid, sulfonic acid, lignosulfonic acid, and combinations or derivatives thereof.

In certain embodiments, the lignin-based enzymatic hydrolysis enhancer further comprises lignosulfonates that are not derived from the lignocellulosic biomass feedstock.

The process further comprises, in some embodiments, applying the lignin-based enzymatic hydrolysis enhancer to a mixture comprising cellulose-rich solids and cellulase enzymes. Any known cellulose-rich solids may be treated, including (but not limited to) cellulose-rich solids obtained from a biomass-pretreatment process selected from the group consisting of steam explosion, hot-water extraction, solvent extraction, acidic solvent extraction, organosolv, dilute-acid pretreatment, ammonia pretreatment, Kraft pulping, sulfite pulping, soda pulping, mechanical pulping, and combinations thereof. In certain embodiments, the cellulose-rich solids are obtained from hardwoods or agricultural residues.

At least 1%, 2%, 3%, 4%, 5%, 6%, 7%, 8%, 9%, or 10% higher glucose yield may be achieved with the lignin-based enzymatic hydrolysis enhancer present in the mixture, compared to an otherwise-identical mixture without the lignin-based enzymatic hydrolysis enhancer.

Optionally, the process further comprises recovering hemicellulosic sugars from the hemicellulose.

The present invention also provides compositions and products. In some embodiments, a lignin-based enzymatic hydrolysis enhancer product is produced by a process as disclosed herein.

A lignin-based enzymatic hydrolysis enhancer comprising partially sulfonated lignin, is provided. Some embodiments provide a lignin-based enzymatic hydrolysis enhancer comprising AVAP® lignin. Certain embodiments provide a lignin-based enzymatic hydrolysis enhancer comprising AVAP® lignin and lignosulfonates.

In some embodiments, the acid is selected from the group consisting of sulfur dioxide, sulfurous acid, sulfur trioxide, sulfuric acid, lignosulfonic acid, and combinations thereof. In certain embodiments, the acid is sulfur dioxide. In step (b), exemplary conditions include SO₂ concentration from about 12 wt % to about 30 wt %, fractionation temperature from about 140° C. to about 170° C., and fractionation time is from about 1 hour to about 2 hours.

The biomass feedstock may be selected from hardwoods, softwoods, forest residues, eucalyptus, industrial wastes, pulp and paper wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof. The process disclosed herein benefits from feedstock flexibility; it is effective for a wide variety of cellulose-containing feedstocks.

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.). Various moisture levels may be associated with the starting biomass. The biomass feedstock need not be, but may be, relatively dry. In general, the biomass is in the form of a particulate or chip, but particle size is not critical in this invention.

Optionally, the process further comprises hydrolyzing cellulose into glucose, and fermenting the glucose to a fermentation product. Optionally, the process further comprises recovering, fermenting, or further treating hemicellulosic sugars derived from the hemicellulose. Optionally, the process further comprises recovering, combusting, or further treating the lignin (i.e., the lignin that is not used for the hydrolysis enhancer).

Glucose that is generated from hydrolysis of amorphous cellulose may be integrated into an overall process to produce ethanol, or another fermentation co-product. Thus in some embodiments, the process further comprises hydrolyzing amorphous cellulose into glucose, and recovering the glucose. The glucose may be purified and sold. Or the glucose may be fermented to a fermentation product, such as but not limited to ethanol. The glucose or a fermentation product may be recycled to the front end, such as to hemicellulose sugar processing, if desired.

When hemicellulosic sugars are recovered and fermented, they may be fermented to produce a monomer or precursor thereof. The monomer may be polymerized to produce a polymer, which may then be combined with the cellulose material to form a polymer-cellulose composite.

In some embodiments, the process further comprises chemically converting the cellulose material to one or more cellulose derivatives. For example, cellulose derivatives may be selected from the group consisting of esters, ethers, ether esters, alkylated compounds, cross-linked compounds, acid-functionalized compounds, base-functionalized compounds, and combinations thereof.

Various types of cellulose functionalization or derivatization may be employed, such as functionalization using polymers, chemical surface modification, functionalization using nanoparticles, modification with inorganics or surfactants, or biochemical modification.

In some embodiments, a first process step is “cooking” (equivalently, “digesting”) which fractionates the three lignocellulosic material components (cellulose, hemicellulose, and lignin) to allow easy downstream removal. Specifically, hemicelluloses are dissolved and over 50% are completely hydrolyzed; cellulose is separated but remains resistant to hydrolysis; and part of the lignin is sulfonated into water-soluble lignosulfonates.

The lignocellulosic material is processed in a solution (cooking liquor) of solvent, water, and acid. The cooking liquor preferably contains at least 10 wt %, such as at least 20 wt %, 30 wt %, 40 wt %, or 50 wt % of a solvent for lignin. For example, the cooking liquor may contain about 30-70 wt % solvent, such as about 50 wt % solvent. The solvent for lignin may be an aliphatic alcohol, such as methanol, ethanol, 1-propanol, 2-propanol, 1-butanol, 2-butanol, isobutanol, 1-pentanol, 1-hexanol, or cyclohexanol. The solvent for lignin may be an aromatic alcohol, such as phenol or cresol. Other lignin solvents are possible, such as (but not limited to) glycerol, methyl ethyl ketone, or diethyl ether. Combinations of more than one solvent may be employed.

Preferably, enough solvent is included in the extractant mixture to dissolve the lignin present in the starting material. The solvent for lignin may be completely miscible, partially miscible, or immiscible with water, so that there may be more than one liquid phase. Potential process advantages arise when the solvent is miscible with water, and also when the solvent is immiscible with water. When the solvent is water-miscible, a single liquid phase forms, so mass transfer of lignin and hemicellulose extraction is enhanced, and the downstream process must only deal with one liquid stream. When the solvent is immiscible in water, the extractant mixture readily separates to form liquid phases, so a distinct separation step can be avoided or simplified. This can be advantageous if one liquid phase contains most of the lignin and the other contains most of the hemicellulose sugars, as this facilitates recovering the lignin from the hemicellulose sugars.

The cooking liquor preferably contains sulfur dioxide and/or sulfurous acid (H₂SO₃). The cooking liquor preferably contains SO₂, in dissolved or reacted form, in a concentration of at least 3 wt %, preferably at least 6 wt %, more preferably at least 8 wt %, such as about 9 wt %, 10 wt %, 11 wt %, 12 wt %, 13 wt %, 14 wt %, 15 wt %, 20 wt %, 25 wt %, 30 wt % or higher. The cooking liquor may also contain one or more species, separately from SO₂, to adjust the pH. The pH of the cooking liquor is typically about 4 or less.

Sulfur dioxide is a preferred acid catalyst, because it can be recovered easily from solution after hydrolysis. The majority of the SO₂ from the hydrolysate may be stripped and recycled back to the reactor. Recovery and recycling translates to less lime required compared to neutralization of comparable sulfuric acid, less solids to dispose of, and less separation equipment. The increased efficiency owing to the inherent properties of sulfur dioxide mean that less total acid or other catalysts may be required. This has cost advantages, since sulfuric acid can be expensive. Additionally, and quite significantly, less acid usage also will translate into lower costs for a base (e.g., lime) to increase the pH following hydrolysis, for downstream operations. Furthermore, less acid and less base will also mean substantially less generation of waste salts (e.g., gypsum) that may otherwise require disposal.

In some embodiments, an additive may be included in amounts of about 0.1 wt % to 10 wt % or more to increase cellulose viscosity. Exemplary additives include ammonia, ammonia hydroxide, urea, anthraquinone, magnesium oxide, magnesium hydroxide, sodium hydroxide, and their derivatives.

The cooking is performed in one or more stages using batch or continuous digestors. Solid and liquid may flow cocurrently or countercurrently, or in any other flow pattern that achieves the desired fractionation. The cooking reactor may be internally agitated, if desired.

Depending on the lignocellulosic material to be processed, the cooking conditions are varied, with temperatures from about 65° C. to 190° C., for example 75° C., 85° C., 95° C., 105° C., 115° C., 125° C., 130° C., 135° C., 140° C., 145° C., 150° C., 155° C., 165° C. or 170° C., and corresponding pressures from about 1 atmosphere to about 15 atmospheres in the liquid or vapor phase. The cooking time of one or more stages may be selected from about 15 minutes to about 720 minutes, such as about 30, 45, 60, 90, 120, 140, 160, 180, 250, 300, 360, 450, 550, 600, or 700 minutes. Generally, there is an inverse relationship between the temperature used during the digestion step and the time needed to obtain good fractionation of the biomass into its constituent parts.

The cooking liquor to lignocellulosic material ratio may be selected from about 1 to about 10, such as about 2, 3, 4, 5, or 6. In some embodiments, biomass is digested in a pressurized vessel with low liquor volume (low ratio of cooking liquor to lignocellulosic material), so that the cooking space is filled with ethanol and sulfur dioxide vapor in equilibrium with moisture. The cooked biomass is washed in alcohol-rich solution to recover lignin and dissolved hemicelluloses, while the remaining pulp is further processed. In some embodiments, the process of fractionating lignocellulosic material comprises vapor-phase cooking of lignocellulosic material with aliphatic alcohol (or other solvent for lignin), water, and sulfur dioxide. See, for example, U.S. Pat. Nos. 8,038,842 and 8,268,125 which are incorporated by reference herein.

A portion or all of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing. Excess sulfur dioxide, following hydrolysis, may be recovered and reused. In some embodiments, sulfur dioxide is saturated in water (or aqueous solution, optionally with an alcohol) at a first temperature, and the hydrolysis is then carried out at a second, generally higher, temperature. In some embodiments, sulfur dioxide is sub-saturated. In some embodiments, sulfur dioxide is super-saturated. In some embodiments, sulfur dioxide concentration is selected to achieve a certain degree of lignin sulfonation, such as 1%, 2%, 3%, 4%, 5%, 6%, 7%, 8%, 9%, or 10% sulfur content. SO₂ reacts chemically with lignin to form stable lignosulfonic acids which may be present both in the solid and liquid phases.

The concentration of sulfur dioxide, additives, and aliphatic alcohol (or other solvent) in the solution and the time of cook may be varied to control the yield of cellulose and hemicellulose in the pulp. The concentration of sulfur dioxide and the time of cook may be varied to control the yield of lignin versus lignosulfonates in the hydrolysate. In some embodiments, the concentration of sulfur dioxide, temperature, and the time of cook may be varied to control the yield of fermentable sugars.

Once the desired amount of fractionation of both hemicellulose and lignin from the solid phase is achieved, the liquid and solid phases are separated. Conditions for the separation may be selected to minimize or enhance the reprecipitation of the extracted lignin on the solid phase. Minimizing lignin reprecipitation is favored by conducting separation or washing at a temperature of at least the glass-transition temperature of lignin (about 120° C.); conversely, enhancing lignin reprecipitation is favored by conducting separation or washing at a temperature less than the glass-transition temperature of lignin.

The physical separation can be accomplished either by transferring the entire mixture to a device that can carry out the separation and washing, or by removing only one of the phases from the reactor while keeping the other phase in place. The solid phase can be physically retained by appropriately sized screens through which liquid can pass. The solid is retained on the screens and can be kept there for successive solid-wash cycles. Alternately, the liquid may be retained and solid phase forced out of the reaction zone, with centrifugal or other forces that can effectively transfer the solids out of the slurry. In a continuous system, countercurrent flow of solids and liquid can accomplish the physical separation.

The recovered solids normally will contain a quantity of lignin and sugars, some of which can be removed easily by washing. The washing-liquid composition can be the same as or different than the liquor composition used during fractionation. Multiple washes may be performed to increase effectiveness. Preferably, one or more washes are performed with a composition including a solvent for lignin, to remove additional lignin from the solids, followed by one or more washes with water to displace residual solvent and sugars from the solids. Recycle streams, such as from solvent-recovery operations, may be used to wash the solids.

After separation and washing as described, a solid phase and at least one liquid phase are obtained. The solid phase contains substantially undigested cellulose. A single liquid phase is usually obtained when the solvent and the water are miscible in the relative proportions that are present. In that case, the liquid phase contains, in dissolved form, most of the lignin originally in the starting lignocellulosic material, as well as soluble monomeric and oligomeric sugars formed in the hydrolysis of any hemicellulose that may have been present. Multiple liquid phases tend to form when the solvent and water are wholly or partially immiscible. The lignin tends to be contained in the liquid phase that contains most of the solvent. Hemicellulose hydrolysis products tend to be present in the liquid phase that contains most of the water.

In some embodiments, hydrolysate from the cooking step is subjected to pressure reduction. Pressure reduction may be done at the end of a cook in a batch digestor, or in an external flash tank after extraction from a continuous digestor, for example. The flash vapor from the pressure reduction may be collected into a cooking liquor make-up vessel. The flash vapor contains substantially all the unreacted sulfur dioxide which may be directly dissolved into new cooking liquor. The cellulose is then removed to be washed and further treated as desired.

A process washing step recovers the hydrolysate from the cellulose. The washed cellulose is pulp that may be used for various purposes (e.g., paper or nanocellulose production). The weak hydrolysate from the washer continues to the final reaction step; in a continuous digestor this weak hydrolysate may be combined with the extracted hydrolysate from the external flash tank. In some embodiments, washing and/or separation of hydrolysate and cellulose-rich solids is conducted at a temperature of at least about 100° C., 110° C., or 120° C. The washed cellulose may also be used for glucose production via cellulose hydrolysis with enzymes or acids.

In another reaction step, the hydrolysate may be further treated in one or multiple steps to hydrolyze the oligomers into monomers. This step may be conducted before, during, or after the removal of solvent and sulfur dioxide. The solution may or may not contain residual solvent (e.g. alcohol). In some embodiments, sulfur dioxide is added or allowed to pass through to this step, to assist hydrolysis. In these or other embodiments, an acid such as sulfurous acid or sulfuric acid is introduced to assist with hydrolysis. In some embodiments, the hydrolysate is autohydrolyzed by heating under pressure. In some embodiments, no additional acid is introduced, but lignosulfonic acids produced during the initial cooking are effective to catalyze hydrolysis of hemicellulose oligomers to monomers. In various embodiments, this step utilizes sulfur dioxide, sulfurous acid, sulfuric acid at a concentration of about 0.01 wt % to 30 wt %, such as about 0.05 wt %, 0.1 wt %, 0.2 wt %, 0.5 wt %, 1 wt %, 2 wt %, 5 wt %, 10 wt %, or 20 wt %. This step may be carried out at a temperature from about 100° C. to 220° C., such as about 110° C., 120° C., 130° C., 140° C., 150° C., 160° C., 170° C., 180° C., 190° C., 200° C., or 210° C. Heating may be direct or indirect to reach the selected temperature.

The reaction step produces fermentable sugars which can then be concentrated by evaporation to a fermentation feedstock. Concentration by evaporation may be accomplished before, during, or after the treatment to hydrolyze oligomers. The final reaction step may optionally be followed by steam stripping of the resulting hydrolysate to remove and recover sulfur dioxide and alcohol, and for removal of potential fermentation-inhibiting side products. The evaporation process may be under vacuum or pressure, from about −0.1 atmospheres to about 10 atmospheres, such as about 0.1 atm, 0.3 atm, 0.5 atm, 1.0 atm, 1.5 atm, 2 atm, 4 atm, 6 atm, or 8 atm.

Recovering and recycling the sulfur dioxide may utilize separations such as, but not limited to, vapor-liquid disengagement (e.g. flashing), steam stripping, extraction, or combinations or multiple stages thereof. Various recycle ratios may be practiced, such as about 0.1, 0.2, 0.3, 0.4, 0.5, 0.6, 0.7, 0.8, 0.9, 0.95, or more. In some embodiments, about 90-99% of initially charged SO₂ is readily recovered by distillation from the liquid phase, with the remaining 1-10% (e.g., about 3-5%) of the SO₂ primarily bound to dissolved lignin in the form of lignosulfonates.

In a preferred embodiment, the evaporation step utilizes an integrated alcohol stripper and evaporator. Evaporated vapor streams may be segregated so as to have different concentrations of organic compounds in different streams. Evaporator condensate streams may be segregated so as to have different concentrations of organic compounds in different streams. Alcohol may be recovered from the evaporation process by condensing the exhaust vapor and returning to the cooking liquor make-up vessel in the cooking step. Clean condensate from the evaporation process may be used in the washing step.

In some embodiments, an integrated alcohol stripper and evaporator system is employed, wherein aliphatic alcohol is removed by vapor stripping, the resulting stripper product stream is concentrated by evaporating water from the stream, and evaporated vapor is compressed using vapor compression and is reused to provide thermal energy.

The hydrolysate from the evaporation and final reaction step contains mainly fermentable sugars but may also contain lignin depending on the location of lignin separation in the overall process configuration. The hydrolysate may be concentrated to a concentration of about 5 wt % to about 60 wt % solids, such as about 10 wt %, 15 wt %, 20 wt %, 25 wt %, 30 wt %, 35 wt %, 40 wt %, 45 wt %, 50 wt % or 55 wt % solids. The hydrolysate contains fermentable sugars.

Fermentable sugars are defined as hydrolysis products of cellulose, galactoglucomannan, glucomannan, arabinoglucuronoxylans, arabinogalactan, and glucuronoxylans into their respective short-chained oligomers and monomer products, i.e., glucose, mannose, galactose, xylose, and arabinose. The fermentable sugars may be recovered in purified form, as a sugar slurry or dry sugar solids, for example. Any known technique may be employed to recover a slurry of sugars or to dry the solution to produce dry sugar solids.

In some embodiments, the fermentable sugars are fermented to produce biochemicals or biofuels such as (but by no means limited to) ethanol, isopropanol, acetone, 1-butanol, isobutanol, lactic acid, succinic acid, or any other fermentation products. Some amount of the fermentation product may be a microorganism or enzymes, which may be recovered if desired.

When the fermentation will employ bacteria, such as Clostridia bacteria, it is preferable to further process and condition the hydrolysate to raise pH and remove residual SO₂ and other fermentation inhibitors. The residual SO₂ (i.e., following removal of most of it by stripping) may be catalytically oxidized to convert residual sulfite ions to sulfate ions by oxidation. This oxidation may be accomplished by adding an oxidation catalyst, such as FeSO4·7H₂O, that oxidizes sulfite ions to sulfate ions. Preferably, the residual SO₂ is reduced to less than about 100 ppm, 50 ppm, 25 ppm, 10 ppm, 5 ppm, or 1 ppm.

In some embodiments, the process further comprises recovering the lignin as a product. The sulfonated lignin may also be recovered as a product. In certain embodiments, the process further comprises combusting or gasifying the sulfonated lignin, recovering sulfur contained in the sulfonated lignin in a gas stream comprising reclaimed sulfur dioxide, and then recycling the reclaimed sulfur dioxide for reuse.

The process lignin separation step is for the separation of lignin from the hydrolysate and can be located before or after the final reaction step and evaporation. If located after, then lignin will precipitate from the hydrolysate since alcohol has been removed in the evaporation step. The remaining water-soluble lignosulfonates may be precipitated by converting the hydrolysate to an alkaline condition (pH higher than 7) using, for example, an alkaline earth oxide, preferably calcium oxide (lime). The combined lignin and lignosulfonate precipitate may be filtered. The lignin and lignosulfonate filter cake may be dried as a co-product or burned or gasified for energy production. The hydrolysate from filtering may be recovered and sold as a concentrated sugar solution product or further processed in a subsequent fermentation or other reaction step.

Native (non-sulfonated) lignin is hydrophobic, while lignosulfonates are hydrophilic. Hydrophilic lignosulfonates may have less propensity to clump, agglomerate, and stick to surfaces. Even lignosulfonates that do undergo some condensation and increase of molecular weight, will still have an HSO₃ group that will contribute some solubility (hydrophilic).

In some embodiments, the soluble lignin precipitates from the hydrolysate after solvent has been removed in the evaporation step. In some embodiments, reactive lignosulfonates are selectively precipitated from hydrolysate using excess lime (or other base, such as ammonia) in the presence of aliphatic alcohol. In some embodiments, hydrated lime is used to precipitate lignosulfonates. In some embodiments, part of the lignin is precipitated in reactive form and the remaining lignin is sulfonated in water-soluble form.

The process fermentation and distillation steps are intended for the production of fermentation products, such as alcohols or organic acids. After removal of cooking chemicals and lignin, and further treatment (oligomer hydrolysis), the hydrolysate contains mainly fermentable sugars in water solution from which any fermentation inhibitors have been preferably removed or neutralized. The hydrolysate is fermented to produce dilute alcohol or organic acids, from 1 wt % to 20 wt % concentration. The dilute product is distilled or otherwise purified as is known in the art.

When alcohol is produced, such as ethanol, some of it may be used for cooking liquor makeup in the process cooking step. Also, in some embodiments, a distillation column stream, such as the bottoms, with or without evaporator condensate, may be reused to wash cellulose. In some embodiments, lime may be used to dehydrate product alcohol. Side products may be removed and recovered from the hydrolysate. These side products may be isolated by processing the vent from the final reaction step and/or the condensate from the evaporation step. Side products include furfural, hydroxymethyl furfural (HMF), methanol, acetic acid, and lignin-erived compounds, for example.

The glucose may be fermented to an alcohol, an organic acid, or another fermentation product. The glucose may be used as a sweetener or isomerized to enrich its fructose content. The glucose may be used to produce baker's yeast. The glucose may be catalytically or thermally converted to various organic acids and other materials.

When hemicellulose is present in the starting biomass, all or a portion of the liquid phase contains hemicellulose sugars and soluble oligomers. It is preferred to remove most of the lignin from the liquid, as described above, to produce a fermentation broth which will contain water, possibly some of the solvent for lignin, hemicellulose sugars, and various minor components from the digestion process. This fermentation broth can be used directly, combined with one or more other fermentation streams, or further treated. Further treatment can include sugar concentration by evaporation; addition of glucose or other sugars (optionally as obtained from cellulose saccharification); addition of various nutrients such as salts, vitamins, or trace elements; pH adjustment; and removal of fermentation inhibitors such as acetic acid and phenolic compounds. The choice of conditioning steps should be specific to the target product(s) and microorganism(s) employed.

In some embodiments, hemicellulose sugars are not fermented but rather are recovered and purified, stored, sold, or converted to a specialty product. Xylose, for example, can be converted into xylitol.

A lignin product can be readily obtained from a liquid phase using one or more of several methods. One simple technique is to evaporate off all liquid, resulting in a solid lignin-rich residue. This technique would be especially advantageous if the solvent for lignin is water-immiscible. Another method is to cause the lignin to precipitate out of solution. Some of the ways to precipitate the lignin include (1) removing the solvent for lignin from the liquid phase, but not the water, such as by selectively evaporating the solvent from the liquid phase until the lignin is no longer soluble; (2) diluting the liquid phase with water until the lignin is no longer soluble; and (3) adjusting the temperature and/or pH of the liquid phase. Methods such as centrifugation can then be utilized to capture the lignin. Yet another technique for removing the lignin is continuous liquid-liquid extraction to selectively remove the lignin from the liquid phase, followed by removal of the extraction solvent to recover relatively pure lignin.

The present invention also provides systems configured for carrying out the disclosed processes, and compositions produced therefrom. Any stream generated by the disclosed processes may be partially or completed recovered, purified or further treated, and/or marketed or sold.

### EXAMPLE

The effect of adding hardwood ethanol-soluble lignin is investigated experimentally, at 4 g/L and 8 g/L concentration of the lignin added to solutions containing washed AVAP® hardwood pulp. The pulp composition is about 85 wt % glucan, 4 wt % xylan, and 10 wt % lignin.

The pulp is hydrolyzed in the laboratory using about 2 wt % of a commercial cellulase enzyme solution at the following conditions: 250 mL shaker flash with air shaker at 150 rpm; 5 wt % solids concentration; temperature 50° C.; pH 5.0 (citrate buffer); and 72 hours hydrolysis.

The glucose yield at 48 hours varied from about 88% to about 98%. Pulps surprisingly responded better to enzymatic reaction when hardwood ethanol-soluble lignin is first added to the hydrolysis container.

Hardwood ethanol-soluble lignin improves the enzymatic digestibility of pulp, giving about 5% increase in glucose yield after 2 days at 4 g/L lignin in the hydrolysate and more than 10% increase in glucose yield after 2 days at 8 g/L lignin in the hydrolysate. Hardwood ethanol-soluble lignin also gives about 4% increase in xylose yield after 2 days at 4 g/L lignin in the hydrolysate and about 8% increase in xylose yield after 2 days at 8 g/L lignin in the hydrolysate.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing a lignin-based enzymatic hydrolysis enhancer, said process comprising:
(a) providing a lignocellulosic biomass feedstock;
(b) fractionating said feedstock in the presence of a sulfur-containing acid, a solvent for lignin, and water, to generate cellulose-rich solids and a liquid containing hemicellulose and lignin;
(c) recovering at least some of said lignin from said solvent; and
(d) generating a lignin-based enzymatic hydrolysis enhancer comprising said lignin recovered in step (c).

2. The process of claim 1, wherein said sulfur-containing acid is selected from the group consisting of sulfur dioxide, sulfur trioxide, sulfurous acid, sulfuric acid, sulfonic acid, lignosulfonic acid, and combinations or derivatives thereof.

3. The process of claim 1, wherein said lignocellulosic biomass feedstock is a hardwood.

4. The process of claim 1, wherein said lignin-based enzymatic hydrolysis enhancer further comprises hydrophilic, sulfur-containing lignin derived from said feedstock and said sulfur-containing acid.

5. The process of claim 1, wherein said lignin-based enzymatic hydrolysis enhancer further comprises lignosulfonates that are not derived from said process.

6. The process of claim 1, said process further comprising (i) applying said lignin-based enzymatic hydrolysis enhancer to a mixture comprising cellulose-rich solids and cellulase enzymes, and (ii) enzymatically hydrolyzing said cellulose-rich solids to generate glucose.

7. The process of claim 6, wherein said cellulose-rich solids are obtained from a biomass-pretreatment process selected from the group consisting of steam explosion, hot-water extraction, solvent extraction, acidic solvent extraction, organosolv, dilute-acid pretreatment, ammonia pretreatment, Kraft pulping, sulfite pulping, soda pulping, mechanical pulping, and combinations thereof.

8. The process of claim 6, wherein said lignin-based enzymatic hydrolysis enhancer is present in said mixture at a concentration of about 1 g/L to about 15 g/L.

9. The process of claim 8, wherein said lignin-based enzymatic hydrolysis enhancer is present in said mixture at a concentration of about 2 g/L to about 10 g/L.

10. The process of claim 6, wherein at least 5% higher glucose yield is achieved with said lignin-based enzymatic hydrolysis enhancer present in said mixture, compared to an otherwise-identical mixture without said lignin-based enzymatic hydrolysis enhancer.

11. The process of claim 10, wherein at least 10% higher glucose yield is achieved with said lignin-based enzymatic hydrolysis enhancer present in said mixture, compared to an otherwise-identical mixture without said lignin-based enzymatic hydrolysis enhancer.

12. The process of claim 1, said process further comprising recovering hemicellulosic sugars from said hemicellulose.

13. A lignin-based enzymatic hydrolysis enhancer product produced by a process comprising the steps of:
(a) providing a lignocellulosic biomass feedstock;
(b) fractionating said feedstock in the presence of a sulfur-containing acid, a solvent for lignin, and water, to generate cellulose-rich solids and a liquid containing hemicellulose and lignin;
(c) recovering at least some of said lignin from said solvent; and
(d) generating a lignin-based enzymatic hydrolysis enhancer comprising said lignin recovered in step (c).

14. A lignin-based enzymatic hydrolysis enhancer composition comprising ethanol-soluble lignin.

15. The lignin-based enzymatic hydrolysis enhancer composition of claim 14, wherein said ethanol-soluble lignin is partially sulfonated.

16. The lignin-based enzymatic hydrolysis enhancer composition of claim 14, wherein said composition further comprises hydrophilic, sulfur-containing lignin.

17. The lignin-based enzymatic hydrolysis enhancer composition of claim 14, wherein said composition further comprises lignosulfonates.

18. A mixture comprising the lignin-based enzymatic hydrolysis enhancer composition of claim 14, said mixture further comprising cellulose-rich solids and cellulase enzymes.

19. The mixture of claim 18, wherein said lignin-based enzymatic hydrolysis enhancer composition is present in said mixture at a concentration of about 1 g/L to about 15 g/L.

20. The mixture of claim 19, wherein said lignin-based enzymatic hydrolysis enhancer composition is present in said mixture at a concentration of about 2 g/L to about 10 g/L.
